(19) United States
(12) Patent Application Publication
(10) Pub. No.: US 2024/0341613 A1
(43) Pub. Date: Oct. 17, 2024

(54) SYSTEM AND METHOD FOR CONTACTLESS VASCULAR FLOW MEASUREMENT

(71) Applicant: ARIZONA BOARD OF REGENTS ON BEHALF OF ARIZONA STATE UNIVERSITY, SCOTTSDALE, AZ (US)

(72) Inventors: Daniel W. Bliss, Phoenix, AZ (US); Yu Rong, Tempe, AZ (US)

(21) Appl. No.: 18/294,757

(22) PCT Filed: Jul. 29, 2022
(86) PCT No.: PCT/US2022/038864

§ 371 Date: Feb. 2, 2024

Related U.S. Application Data
(60) Provisional application No. 63/229,833, filed on Aug. 5, 2021.

(51) Int. Cl. A61B 5/0265; A61B 5/00; A61B 5/024; A61B 5/0507

(52) U.S. Cl. CPC A61B 5/0265; A61B 5/0053; A61B 5/02416; A61B 5/0507; A61B 5/7246; A61B 2560/0462

(57) ABSTRACT

System and method for contactless vascular flow measurement are provided. Herein, a measurement circuit emits a radio frequency (RF) waveform toward a human peripheral body part and measures micro-vessel motion in an area of interest of the human peripheral body part based on reflections of the RF waveform. Accordingly, the measurement circuit can extrapolate vascular flow information in the human peripheral body part based on the measured micro-vessel motion. In a non-limiting example, the measurement circuit can detect inner organ vibrations to measure pulse rate, strength, and/or pressure caused by radial arterial blood flow changes at the human peripheral body part. By detecting and measuring the vascular flow via the RF waveform, the vascular flow measurement system provides a non-invasive approach to detect and/or prevent peripheral artery diseases. Further, the measurement circuit emits a very low power, non-ionizing RF waveform to help minimize potential health risks to a human body.

### RELATED APPLICATION

The application claims the benefit of provisional patent application Ser. No. 63/229,833, filed Aug. 5, 2021, the disclosure of which is hereby incorporated herein by reference in its entirety.

### FIELD OF THE DISCLOSURE

The present disclosure relates to remote vital sign detection, and in particular to remote vascular flow (a.k.a. blood flow) sensing at a peripheral body part.

### BACKGROUND

The human vascular system, also referred to as the circulatory system, is made of the vessels that carry blood and other essential fluids through a human body. Blood pumped out by a human heart moves through the vascular system. The vascular system functions as an important component of other body systems, such as the respiratory system and the digestive system. Organs and other body structures may be damaged by vascular disease because of decreased or completely blocked blood flow. The causes of vascular disease, such as blood clots, inflammation, trauma and so on, are not uncommon among people. In this regard, it is important in many health contexts to be able to monitor vascular blood flow.

### SUMMARY

Aspects disclosed in the detailed description include a system and method for contactless vascular flow measurement. In embodiments disclosed herein, a measurement circuit emits a radio frequency (RF) waveform (e.g., a radar waveform) toward a human peripheral body part (e.g., wrist) and measures micro-vessel motion (a.k.a. pressure change) in an area of interest of the human peripheral body part based on reflections of the RF waveform. Accordingly, the measurement circuit can extrapolate vascular flow information in the human peripheral body part based on the measured micro-vessel motion. In a non-limiting example, the measurement circuit can detect inner organ vibrations to measure pulse rate, strength, and/or pressure caused by radial arterial blood flow changes at the human peripheral body part. By detecting and measuring the vascular flow via the RF waveform, the vascular flow measurement system provides a non-invasive approach to detect and/or prevent peripheral artery diseases. Further, unlike conventional computed tomography (CT) and X-rays, the measurement circuit emits a very low power, non-ionizing RF waveform to help minimize potential health risks to a human body.

In one aspect, a vascular flow measurement system is provided. The vascular flow measurement system includes a measurement circuit placed at a distance from a body part under measurement. The measurement circuit includes an emitter circuit. The emitter circuit is configured to emit an RF waveform toward the body part. The measurement circuit also includes a receiver circuit. The receiver circuit is configured to absorb one or more reflections of the RF waveform reflected by the body part. The measurement circuit also includes a processing circuit. The processing circuit is configured to measure a micro-vessel motion in a region of interest of the body part based on the one or more reflections of the RF waveform. The processing circuit is also configured to characterize vascular flow in the region of interest of the body part based on the measured micro-vessel motion.

In another aspect, a method for performing a vascular flow measurement is provided. The method includes emitting an RF waveform toward a body part under measurement. The method also includes absorbing one or more reflections of the RF waveform reflected by the body part. The method also includes measuring a micro-vessel motion in a region of interest of the body part based on the one or more reflections of the RF waveform. The method also includes characterizing vascular flow in the region of interest of the body part based on the measured micro-vessel motion.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1 is a schematic diagram of an exemplary vascular flow measurement system wherein a measurement circuit is configured according to embodiments of the present disclosure to perform contactless vascular flow measurement on a body part;

FIG. 2 is a graphic representation of a sawtooth frequency-modulated continuous-wave (FMCW) waveform that may be emitted by the measurement circuit in FIG. 1 to perform contactless vascular flow measurement on the body part;

FIGS. 3A-3D are graphic diagrams illustrating a process for determining a pulse sensitivity map;

FIG. 4 is a flowchart of an exemplary process that can be employed by the measurement circuit in FIG. 1 to perform contactless vascular flow measurement;

FIG. 5 is a graphical representation of a radar-measured radial arterial pulse waveform;

FIG. 6 is a graphical representation of a radar-measured arterial pulse rate variability;

FIG. 7 is a graphical representation of a radar-measured relative spectral magnitude as a result of varying applied external pressure;

FIG. 8 is a graphical representation of a radar-measured differential

pulse strength/pressure measurement as a result of varying applied external pressure; and

FIG. 9 is a block diagram of the measurement circuit in FIG. 1 according to embodiments disclosed herein.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Aspects disclosed in the detailed description include a system and method for contactless vascular flow measurement. In embodiments disclosed herein, a measurement circuit emits a radio frequency (RF) waveform (e.g., a radar waveform) toward a human peripheral body part (e.g., wrist) and measures micro-vessel motion (a.k.a. pressure change) in an area of interest of the human peripheral body part based on reflections of the RF waveform. Accordingly, the measurement circuit can extrapolate vascular flow information in the human peripheral body part based on the measured micro-vessel motion. In a non-limiting example, the measurement circuit can detect inner organ vibrations to measure pulse rate, strength, and/or pressure caused by radial arterial blood flow changes at the human peripheral body part. By detecting and measuring the vascular flow via the RF waveform, the vascular flow measurement system provides a non-invasive approach to detect and/or prevent peripheral artery diseases. Further, unlike conventional computed tomography (CT) and X-rays, the measurement circuit emits a very low power, non-ionizing RF waveform to help minimize potential health risks to a human body.

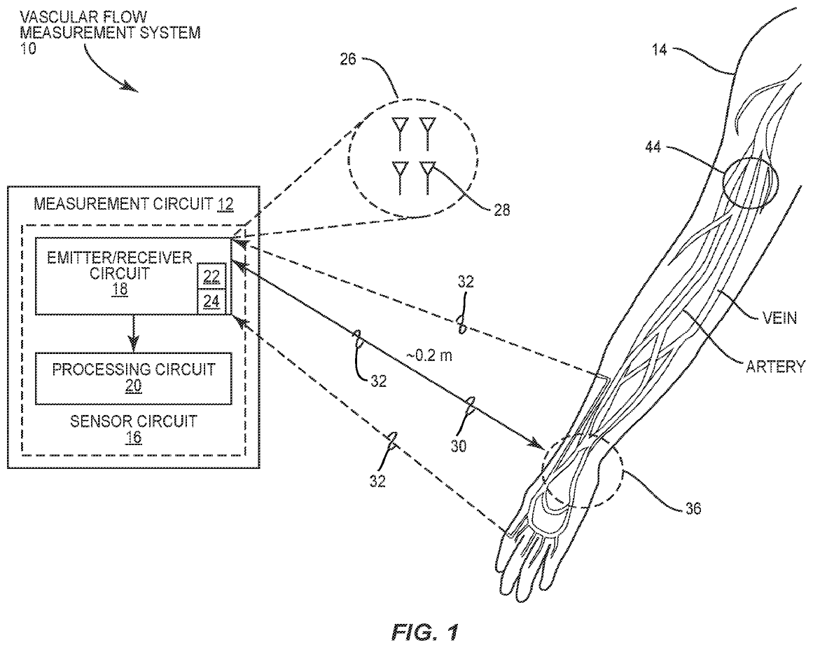

FIG. 1 is a schematic diagram of an exemplary vascular flow measurement system 10 wherein a measurement circuit 12 is configured according to embodiments of the present disclosure to perform contactless vascular flow measurement on a body part 14. In a non-limiting example, the body part 14 can be a human peripheral body part, including but not limited to an arm, a wrist, any hand fingers, a leg, an ankle, any toe fingers, a neck, and a shoulder. Although the embodiments are described herein with respect to a human peripheral body part, it should be appreciated that the system and method disclosed herein may also be applicable to animal body parts.

The measurement circuit 12, is provided at a distance (e.g., two-tenth of a meter) from the body part 14, without contacting the body part 14. The measurement circuit 12 includes a sensor circuit 16. According to an embodiment of the present disclosure, the sensor circuit 16 further includes an emitter/receiver circuit 18 and a processing circuit 20. Specifically, the emitter/receiver circuit 18 can include an emitter circuit 22 and a receiver circuit 24 that are provided in a same die or different dies. The emitter/receiver circuit 18 can also include a power management integrated circuit (PMIC), power amplifier, low-noise amplifier (LNA), transmit/receive filter circuit, RF frontend circuit, and/or antenna switch circuit, which are not shown herein for the sake of simplicity and conciseness. The emitter/receiver circuit 18 can further include an antenna circuit 26, which can include one or more antennas 28. In this regard, the emitter/receiver circuit 18 will be capable of emitting and/or receiving RF signals via such advanced techniques as RF beamforming and multiple-input multiple-output (MIMO) to help boost signal-to-noise ratio (SNR) in the emitted and/or received RF signals.

In contrast to conventional non-contact blood flow measurement approaches, such as optical vision-based systems, which requires proper lighting conditions and skin exposure, the vascular flow measurement system 10 described herein is able to function in dark conditions and penetrate through clothing without being affected by skin tone.

According to an embodiment of the present disclosure, the emitter/receiver circuit 18 is configured to emit an RF waveform 30 toward the body part 14 under measurement. In a non-limiting example, the RF waveform 30 can be a millimeter wave (mmWave) radar waveform, a Terahertz radar waveform, an ultra-wideband (UWB) waveform, or a frequency-modulated continuous-wave (FMCW) waveform.

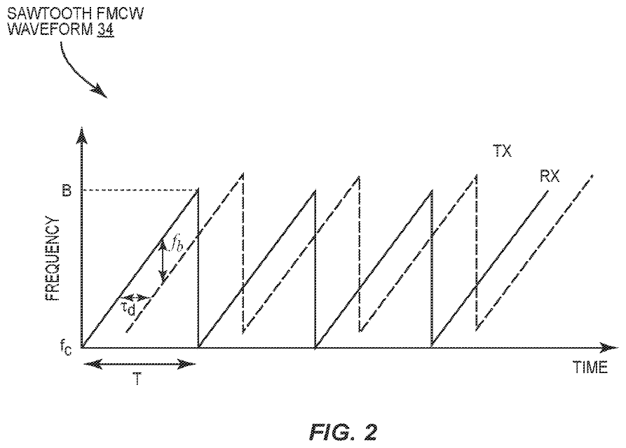

FIG. 2 is a graphical representation of a sawtooth FMCW waveform 34 that may be emitted by the emitter circuit 22 in FIG. 1 as a form of the RF waveform 30. The sawtooth FMCW waveform 34 may be transmitted at an initial frequency f_{c} and increases with sweep time (T) to a maximum frequency fb that is bounded by a signal bandwidth (B). A chirp rate is defined as a speed of the frequency change, which is α constant a within T, α=B/T. In an embodiment, a beamforming technique is applied to boost the received SNR when multiple antennas 28 are provided in the antenna circuit 26.

With reference back to FIG. 1, the body part 14 can cause one or more reflections 32 of the RF beamform 30 toward the measurement circuit 12. As such, the receiver circuit 24 is configured to absorb the reflections 32 of the RF waveform 30 reflected by the body part 14. The processing circuit 20, which can be a field-programmable gate array (FPGA), or an application-specific integrated circuit (ASIC), as an example, is configured to measure a micro-vessel motion (a.k.a. pressure change) in a region of interest 36 (e.g., a wrist) of the body part 14 based on the reflections 32 of the RF waveform 30. Accordingly, the processing circuit 20 can characterize vascular flow in the region of interest 36 of the body part 14 based on the measured micro-vessel motion.

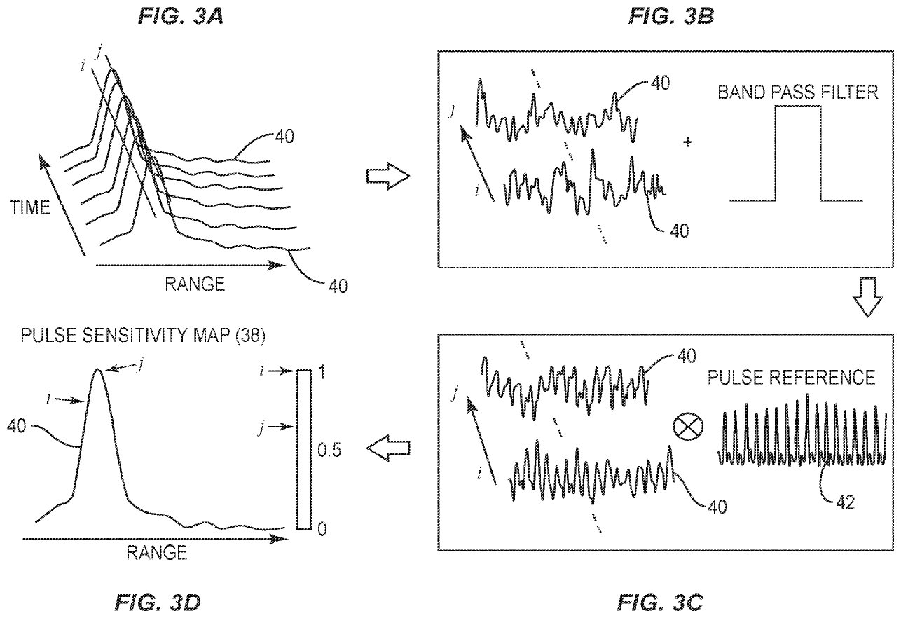

In an embodiment, the processing circuit 20 may determine the region of interest 36 of the body part 14 based on a pulse sensitivity map. In this regard, FIGS. 3A-3D are graphic diagrams illustrating a process that can be employed by the processing circuit 20 in FIG. 1 for determining a pulse sensitivity map 38.

In some embodiments, a wideband signal waveform is used to differentiate the target of interest from the uninteresting background. Usually, a strongest reflection point in a range bin can be used to locate the region of interest 36. However, the pulse signal quality is not necessarily the best at this single range bin. In this regard, the processing circuit 20 is configured to determine the pulse sensitivity map 38 by applying multi-channel signal processing to different phase signals from multiple range bins 40. FIG. 3A provides an exemplary illustration of the range bins 40.

In a non-limiting example, 20-seconds of the RF waveform 30 are used in conjunction with a pulse reference signal 42 to determine the pulse sensitivity map 38. According to an embodiment of the present disclosure, the pulse reference signal 42 may be generated using a fingertip oximeter.

As shown in FIG. 3B, a temporal phase variation is first extracted from each of the range bins 40. Next, as shown in FIG. 3C, a cross-correlation between the extracted temporal phase and the reference pulse signal are computed. Finally, as shown in FIG. 3D, the computed correlation values are color coded and overlaid with one of the range bins 40 from the 20 seconds of data. The darker color indicates the higher pulse sensitivity is in this range bin, and vice versa.

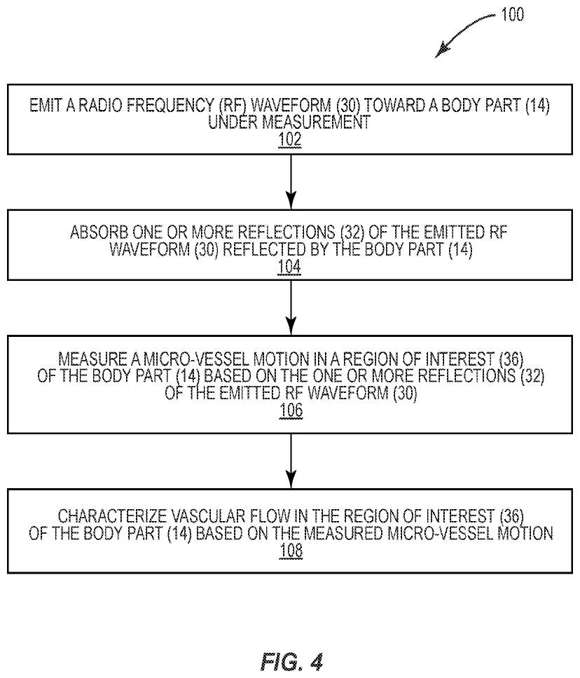

The measurement circuit 12 can be configured to perform contactless vascular flow measurement based on a process. In this regard, FIG. 4 is a flowchart of an exemplary process 100 that can be employed by the measurement circuit 12 in FIG. 1 to perform contactless vascular flow measurement.

First in the process 100, the emitter circuit 22 emits the RF waveform 30 toward the body part 14 under measurement (step 102). Next in the process 100, the receiver circuit 24 absorbs the reflections 32 of the emitted RF waveform 30 reflected by the body part 14 (step 104). Next in the process 100, the processing circuit 20 measures the micro-vessel motion in the region of interest 36 of the body part 14 based on the reflections 32 of the emitted RF waveform 30 (step 106). Next in the process 100, the processing circuit 20 characterizes vascular flow in the region of interest 36 of the body part 14 based on the measured micro-vessel motion (step 108). In a non-limiting example, the measured vascular flow can include a pulse signal, a heart rate, a heart rate variability, and/or a blood pressure.

With reference back to FIG. 1, the emitter circuit may be a mmWave or Terahertz wave radar system with a boresight of the antenna circuit 26 aligned with the region of interest 36. An arm pressure pump may be used as an external force to apply pressure at an upper arm location 44 to change arm blood flow and consequently radial arterial pulse pressure. A hand stabling platform may be designed to reduce or avoid hand movements during the contactless vascular flow measurement. The region of interest 36 (a.k.a. the measurement site) may be pre-determined by wrist palpation, which is a common way to feel a pulse.

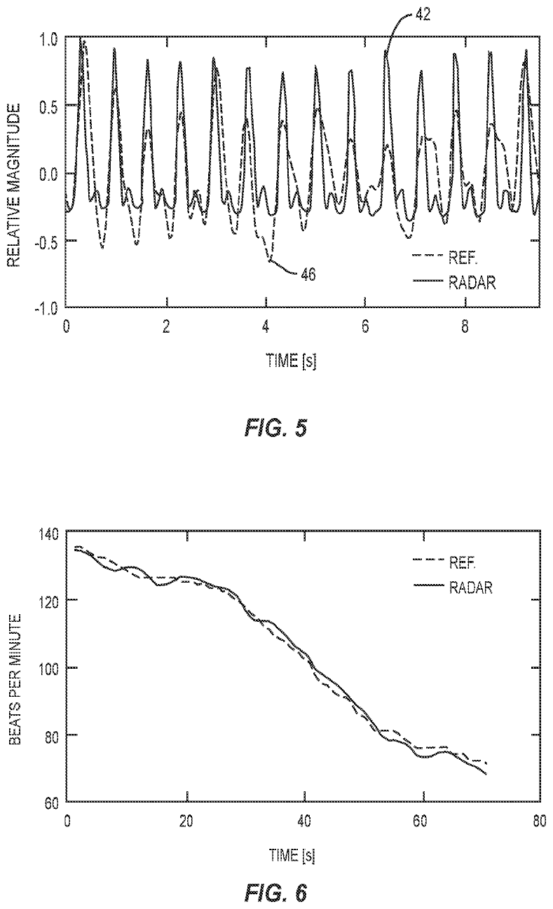

FIG. 5 is a graphical representation of a radar-measured radial arterial pulse waveform 46. In this example, the surface wrist skin motion is extracted from the radar phase using a signal processing method. One chunk of measurement is displayed in FIG. 6 compared with the pulse reference signal 42. According to FIG. 5, the radar-measured radial arterial pulse waveform 46 resembles the pulse reference signal 42.

FIG. 6 is a graphical representation of a radar-measured arterial pulse rate variability. In this example, the pulse rate variability is analyzed for a radial artery using radar. Herein, the vascular flow measurement is conducted in a similar setting as illustrated in FIG. 1. However, the body part 14 under measurement performs a 10-minute cardio physical exercise to elevate heart rate right before the vascular flow measurement is taken. As such, FIG. 6 shows a decreasing trend of a relaxing heart rate (e.g., during cooldown). The radar measurement follows this trend and matches with the heart rate calculated from the pulse reference signal 42.

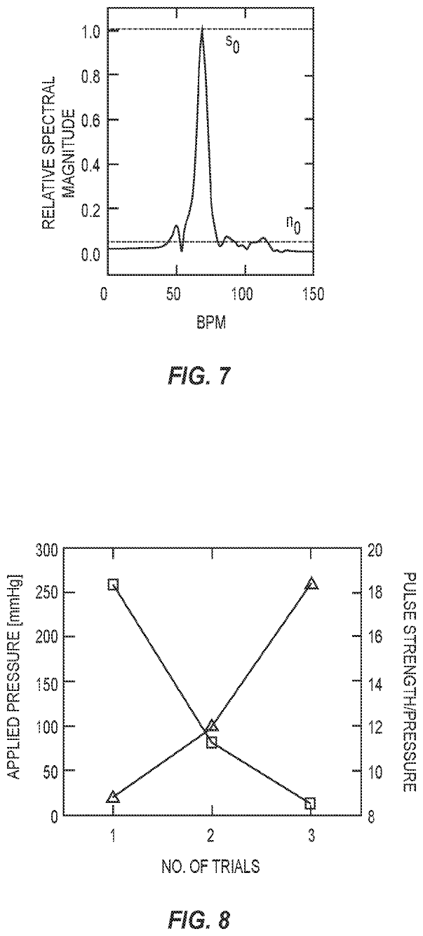

FIG. 7 is a graphical representation of a radar-measured relative spectral magnitude as a result of varying applied external pressure. FIG. 8 is a graphical representation of a radar-measured differential pulse strength/pressure measurement as a result of varying applied external pressure.

Herein, a differential radial arterial pulse pressure is measured. Absolute blood pulse pressure measurement usually requires an external calibration process like those seen in conventional cuff-based blood pressure devices. Indirectly, pulse strength variation is used as a metric to mimic the blood pressure variation because of the direct relationship between the measured pulse strength and the blood pressure.

The pulse strength is empirically quantified as the ratio of the peak spectral value to the estimated noise floor. Three measurements were taken for this demonstration. At each measurement, a different external arm pressure was applied and therefore resulted in a different arterial pulse strength/pressure. A strong negative correlation is shown among the exerted external pressure and the measured arterial pulse strength/pressure. As the external pressure increases at the upper arm, the radial arterial pulse pressure taken at the same arm decreases. This result is consistent with expectations as the high pressure blocks the blood flow and reduces the pulsation at the subject's wrist.

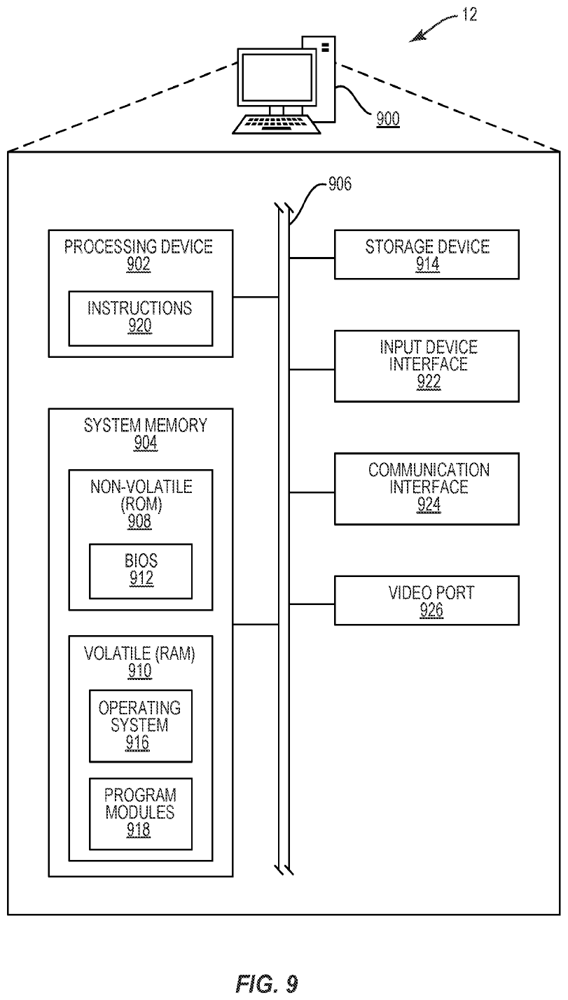

FIG. 9 is a block diagram of the measurement circuit 12 according to embodiments disclosed herein. The measurement circuit 12 and, more specifically the processing circuit 20, includes or is implemented as a computer system 900, which comprises any computing or electronic device capable of including firmware, hardware, and/or executing software instructions that could be used to perform any of the methods or functions described above. In this regard, the computer system 900 may be a circuit or circuits included in an electronic board card, such as a printed circuit board (PCB), a server, a personal computer, a desktop computer, a laptop computer, an array of computers, a personal digital assistant (PDA), a computing pad, a mobile device, or any other device, and may represent, for example, a server or a user's computer.

The exemplary computer system 900 in this embodiment includes a processing device 902 or processor, a system memory 904, and a system bus 906. The system memory 904 may include non-volatile memory 908 and volatile memory 910. The non-volatile memory 908 may include read-only memory (ROM), erasable programmable read-only memory (EPROM), electrically erasable programmable read-only memory (EEPROM), and the like. The volatile memory 910 generally includes random-access memory (RAM) (e.g., dynamic random-access memory (DRAM), such as synchronous DRAM (SDRAM)). A basic input/output system (BIOS) 912 may be stored in the non-volatile memory 908 and can include the basic routines that help to transfer information between elements within the computer system 900.

The system bus 906 provides an interface for system components including, but not limited to, the system memory 904 and the processing device 902. The system bus 906 may be any of several types of bus structures that may further interconnect to a memory bus (with or without a memory controller), a peripheral bus, and/or a local bus using any of a variety of commercially available bus architectures.

The processing device 902 represents one or more commercially available or proprietary general-purpose processing devices, such as a microprocessor, central processing unit (CPU), or the like. More particularly, the processing device 902 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 902 is configured to execute processing logic instructions for performing the operations and steps discussed herein.

In this regard, the various illustrative logical blocks, modules, and circuits described in connection with the embodiments disclosed herein may be implemented or performed with the processing device 902, which may be a microprocessor, FPGA, a digital signal processor (DSP), an ASIC, or other programmable logic device, a discrete gate or transistor logic, discrete hardware components, or any combination thereof designed to perform the functions described herein. Furthermore, the processing device 902 may be a microprocessor, or may be any conventional processor, controller, microcontroller, or state machine. The processing device 902 may also be implemented as a combination of computing devices (e.g., a combination of a DSP and a microprocessor, a plurality of microprocessors, one or more microprocessors in conjunction with a DSP core, or any other such configuration).

The computer system 900 may further include or be coupled to a non-transitory computer-readable storage medium, such as a storage device 914, which may represent an internal or external hard disk drive (HDD), flash memory, or the like. The storage device 914 and other drives associated with computer-readable media and computer-usable media may provide non-volatile storage of data, data structures, computer-executable instructions, and the like. Although the description of computer-readable media above refers to an HDD, it should be appreciated that other types of media that are readable by a computer, such as optical disks, magnetic cassettes, flash memory cards, cartridges, and the like, may also be used in the operating environment, and, further, that any such media may contain computer-executable instructions for performing novel methods of the disclosed embodiments.

An operating system 916 and any number of program modules 918 or other applications can be stored in the volatile memory 910, wherein the program modules 918 represent a wide array of computer-executable instructions corresponding to programs, applications, functions, and the like that may implement the functionality described herein in whole or in part, such as through instructions 920 on the processing device 902. The program modules 918 may also reside on the storage mechanism provided by the storage device 914. As such, all or a portion of the functionality described herein may be implemented as a computer program product stored on a transitory or non-transitory computer-usable or computer-readable storage medium, such as the storage device 914, the non-volatile memory 908, the volatile memory 910, the instructions 920, and the like. The computer program product includes complex programming instructions, such as a complex computer-readable program code, to cause the processing device 902 to carry out the steps necessary to implement the functions described herein.

An operator, such as the user, may also be able to enter one or more configuration commands to the computer system 900 through a keyboard, a pointing device such as a mouse, or a touch-sensitive surface, such as the display device, via an input device interface 922 or remotely through a web interface, terminal program, or the like via a communication interface 924. The communication interface 924 may be wired or wireless and facilitate communications with any number of devices via a communications network in a direct or indirect fashion. An output device, such as a display device, can be coupled to the system bus 906 and driven by a video port 926. Additional inputs and outputs to the computer system 900 may be provided through the system bus 906 as appropriate to implement embodiments described herein.

The operational steps described in any of the exemplary embodiments herein are described to provide examples and discussion. The operations described may be performed in numerous different sequences other than the illustrated sequences. Furthermore, operations described in a single operational step may actually be performed in a number of different steps. Additionally, one or more operational steps discussed in the exemplary embodiments may be combined.

Those skilled in the art will recognize improvements and modifications to the preferred embodiments of the present disclosure. All such improvements and modifications are considered within the scope of the concepts disclosed herein and the claims that follow.

1. A vascular flow measurement system comprising:
a measurement circuit placed at a distance from a body part under measurement, the measurement circuit comprising:
an emitter circuit configured to emit a radio frequency, RF, waveform toward the body part;
a receiver circuit configured to absorb one or more reflections of the emitted RF waveform reflected by the body part; and
a processing circuit configured to:
measure a micro-vessel motion in a region of interest of the body part based on the one or more reflections of the emitted RF waveform; and
characterize vascular flow in the region of interest of the body part based on the measured micro-vessel motion.

2. The vascular flow measurement system of claim 1, wherein a characterized level of the vascular flow is positively related to a measured strength of the micro-vessel motion.

3. The vascular flow measurement system of claim 1, wherein the emitter circuit is further configured to emit the RF waveform as one of: a millimeter wave radar waveform, a Terahertz radar waveform, an ultra-wideband, UWB, waveform, and a sawtooth frequency-modulated continuous-wave, FMCW, waveform.

4. The vascular flow measurement system of claim 1, wherein the body part comprises a human peripheral body part.

5. The vascular flow measurement system of claim 4, wherein the human peripheral body part comprises a human wrist.

6. The vascular flow measurement system of claim 1, wherein the processing circuit is further configured to determine the region of interest of the body part based on a pulse sensitivity map.

7. The vascular flow measurement system of claim 6, wherein the processing circuit is further configured to:
extract a respective one of a plurality of temporal phase variations from a respective one of a plurality of range bins;
compute a cross-correlation value between each of the plurality of temporal phase variations and a reference pulse signal; and
overlay the cross-correlation value computed for each of the plurality of temporal phase variations with one of the plurality of range bins to generate the pulse sensitivity map.

8. The vascular flow measurement system of claim 1, wherein the emitter circuit comprises a plurality of antennas configured to emit the RF waveform via RF beamforming.

9. A method for performing a vascular flow measurement comprising:
emitting radio frequency, RF, waveform toward a body part under measurement;
absorbing one or more reflections of the emitted RF waveform reflected by the body part;
measuring a micro-vessel motion in a region of interest of the body part based on the one or more reflections of the emitted RF waveform; and
characterizing vascular flow in the region of interest of the body part based on the measured micro-vessel motion.

10. The method of claim 9, wherein a characterized level of the vascular flow is positively related to a measured strength of the micro-vessel motion.

11. The method of claim 9, wherein emitting the RF waveform comprises emitting the RF waveform as one of: a millimeter wave radar waveform, a Terahertz radar waveform, an ultra-wideband, UWB, waveform, and a sawtooth frequency-modulated continuous-wave, FMCW, waveform.

12. The method of claim 9, wherein emitting the RF waveform toward the body part comprises emitting the RF waveform toward a human peripheral body part.

13. The method of claim 12, wherein emitting the RF waveform toward the human peripheral body part comprises emitting the RF waveform toward a human wrist.

14. The method of claim 9, further comprising determining the region of interest of the body part based on a pulse sensitivity map.

15. The method of claim 14, further comprising:
extracting a respective one of a plurality of temporal phase variations from a respective one of a plurality of range bins;
computing a cross-correlation value between each of the plurality of temporal phase variations and a reference pulse signal; and
overlaying the cross-correlation value computed for each of the plurality of temporal phase variations with one of the plurality of range bins to generate the pulse sensitivity map.

16. The method of claim 15, further comprising generating the reference pulse signal using a fingertip oximeter.

17. The method of claim 9, further comprising:
generating the RF waveform using a radar that is one of a millimeter wave and a Terahertz wave radar; and
changing the vascular flow in the body part using a pressure pump.

18. The method of claim 17, further comprising:
determining a measurement site on the body part via wrist palpation; and
aligning boresight of the radar with the measurement site on the body part.

19. The method of claim 9, further comprising stabilizing the body part during the contactless vascular flow measurement.

20. The method of claim 9, wherein emitting the RF waveform comprises emitting the RF waveform via RF beamforming.
